(19) United States
(12) Patent Application Publication
(10) Pub. No.: US 2026/0315689 A1
(43) Pub. Date: Oct. 15, 2026

(54) CLOSED SUBCUTANEOUS CAVITY IRRIGATION METHODS AND SYSTEMS

(71) Applicant: BOARD OF REGENTS, THE UNIVERSITY OF TEXAS SYSTEM, Austin, TX (US)

(72) Inventors: Justin Earl BIRD, Houston, TX (US); Roland VON KURNATOWSKI, Houston, TX (US); Jacob PIERCE, Houston, TX (US); Jacob RHINES, Houston, TX (US); Savannah Esteve MORREALE, Houston, TX (US)

(21) Appl. No.: 19/484,416

(22) PCT Filed: May 22, 2024
(86) PCT No.: PCT/US2024/030550

§ 371 Date: Nov. 14, 2025

Related U.S. Application Data
(60) Provisional application No. 63/503,591, filed on May 22, 2023.

(51) Int. Cl. A61M 5/142; A61M 5/168; A61M 25/00

(52) U.S. Cl. CPC A61M 5/14212; A61M 5/14244; A61M 5/16813; A61M 25/0026

(57) ABSTRACT

Systems and methods preventing, treating, or otherwise addressing infection risk in a closed subcutaneous cavity. In one example, a method includes: (a) implanting an elongated fluid conduit such that the fluid conduit is in fluid communication with the closed subcutaneous cavity; (b) operating an irrigation system in accordance with an irrigation treatment program including: (i) pumping the irrigation fluid from an irrigation fluid source through the fluid conduit and into the closed subcutaneous cavity in accordance with a parameter of the irrigation treatment program; (ii) allowing the irrigation fluid to dwell in the closed subcutaneous cavity in accordance with another parameter; and (iii) pumping the irrigation fluid from the closed subcutaneous cavity through the fluid conduit in accordance with another parameter.

### CROSS REFERENCE TO RELATED APPLICATIONS

This application claims priority to and the benefit of the filing date of U.S. provisional patent application Ser. No. 63/503,591 filed May 22, 2023 for “Closed Subcutaneous Cavity Irrigation Methods And Systems,” the entire contents of which is hereby incorporated by reference.

### RELATED FIELDS

Irrigation methods and systems, particularly irrigation methods and systems for irrigating closed subcutaneous cavities to prevent, treat, or otherwise address infection risks. Irrigation includes lavage, such as, but not limited to, washing by flushing with a fluid.

### BACKGROUND

Joint infections are common, costly, and highly morbid, often requiring repeat surgeries, long courses of antibiotics, long hospital stays, and potentially amputations. Treatment often includes surgical procedures to irrigate and debride infected tissues. In the case of infected joint replacements, implants may need to be removed and re-implanted once the infection is cleared. Inherent delays in diagnosis to treatment exacerbate the problem. Clinicians may aspirate a joint in the clinic and preemptively start oral antibiotics, however, there is no standardized method/device for the purpose of repetitively aspirating and irrigating the joint.

Other types of deep infections provide similar challenges. Deep infections can create abscess cavities that are difficult to treat with intravenous antibiotics alone. Deep infections often require repeat incision and copious irrigation of the infected area. Multiple surgical procedures increase risk of complications and length of stay; therefore, Interventional Radiologist are often asked to percutaneously place indwelling catheters into difficult to reach abscess cavities in order to drain the infection hoping to obviate the need for major surgery. Percutaneous catheter placement does not currently include methods for irrigation; drainage alone may prolong the infection course.

### SUMMARY

We have invented improved systems and methods for preventing, treating, or otherwise addressing infection risk sin closed subcutaneous cavities.

In one example, a method of preventing, treating, or otherwise addressing infection risk in a closed subcutaneous cavity using an irrigation system involves implanting an elongated fluid conduit such that the fluid conduit is in fluid communication with the closed subcutaneous cavity. Next, the irrigation system is operated in accordance with an irrigation treatment program, the irrigation treatment program including at least one parameter for pumping an irrigation fluid into the closed subcutaneous cavity, at least one parameter for dwell time of the irrigation fluid in the closed subcutaneous cavity, and at least one parameter for pumping the irrigation fluid from the closed subcutaneous cavity. Operating the irrigation system in accordance with the irrigation treatment program includes: (i) pumping the irrigation fluid from an irrigation fluid source through the fluid conduit and into the closed subcutaneous cavity in accordance with the parameter for pumping the irrigation fluid into the closed subcutaneous cavity; (ii) allowing the irrigation fluid to dwell in the closed subcutaneous cavity in accordance with the parameter for dwell time; and (iii) pumping the irrigation fluid from the closed subcutaneous cavity through the fluid conduit in accordance with the parameter for pumping the irrigation fluid from the closed subcutaneous cavity.

In this example, the irrigation system may include a re-configurable pump and valve arrangement. During pumping of the irrigation fluid from the irrigation fluid source into the closed subcutaneous cavity, the irrigation system may be configured to cause the re-configurable pump and valve arrangement to be in a first state in which fluid flow between the irrigation fluid source and the pump is allowed and fluid flow between the pump and an irrigation fluid outlet is prevented. During pumping of the irrigation fluid from the closed subcutaneous cavity to the irrigation fluid outlet the irrigation system may be configured to cause the re-configurable pump and valve arrangement to be in a second state in which fluid flow between the irrigation fluid source and the pump is prevented and fluid flow between the pump and the irrigation fluid outlet is allowed.

In this example, the elongated fluid conduit may be a multi-lumen catheter. One lumen may be configured to convey the irrigation fluid (and optionally a medicament) to and from the closed subcutaneous cavity. Additional lumens may be configured to convey ultrasound and antibiotic light to the closed subcutaneous cavity.

### BRIEF DESCRIPTION OF THE FIGURES

FIG. 1 shows an example of an irrigation system.

FIG. 2 shows an example of a multi-lumen catheter.

FIG. 3 shows an example of a wearable irrigation system.

FIG. 4 shows an example of an irrigation method.

FIG. 5 shows an example of an irrigation system including a passive drainage functionality.

FIGS. 6-9 illustrate a cross-contamination test setup and results.

### DETAILED DESCRIPTION

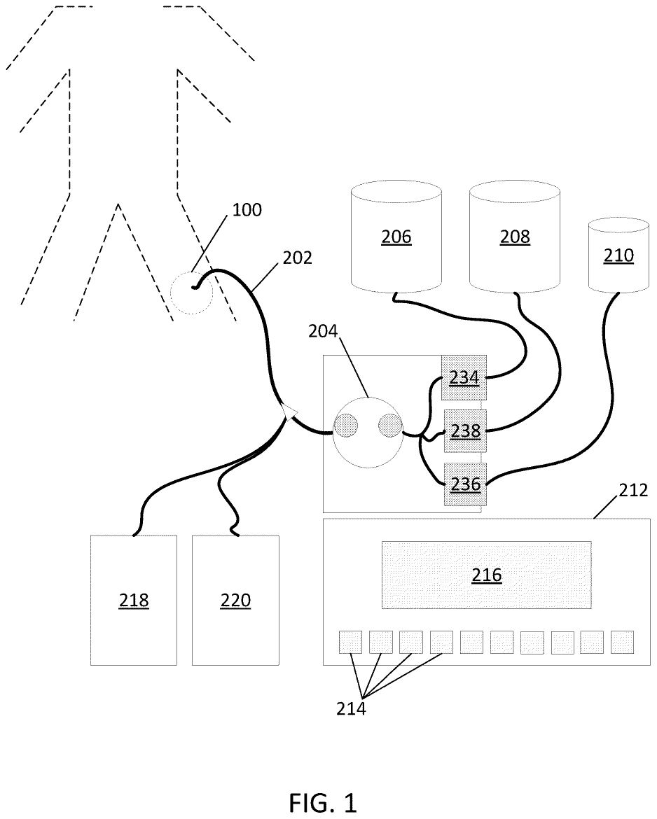

FIG. 1 shows an example of an irrigation system for preventing, treating, or otherwise addressing infection risk in a closed subcutaneous cavity 100. In some uses, the closed subcutaneous cavity 100 may be a joint capsule, a seroma, an abscess, surgical defect, or a subcutaneous wound. The irrigation system is for performing non-surgical lavages, washouts, and other irrigations of the closed subcutaneous cavity 100.

In the example of FIG. 1 the irrigation system includes an elongated fluid conduit 202 that is configured for implantation in the closed subcutaneous cavity 100, a pump 204 that is configured to pump an irrigation fluid through the elongated fluid conduit 202, an irrigation fluid source 206 for supplying irrigation fluid to the pump 204, a fluid outlet container 208 for receiving used irrigation fluid pumped out of the closed subcutaneous cavity 100, and a medicament source 210 for supplying a medicament to the pump 204. In some instances, the medicament may be an antibiotic or an anti-biofilm agent. Anti-biofilm agents may include, for example, natural anti-biofilm agents such as phytochemicals, biosurfactants, antimicrobial peptides, and microbial enzymes.

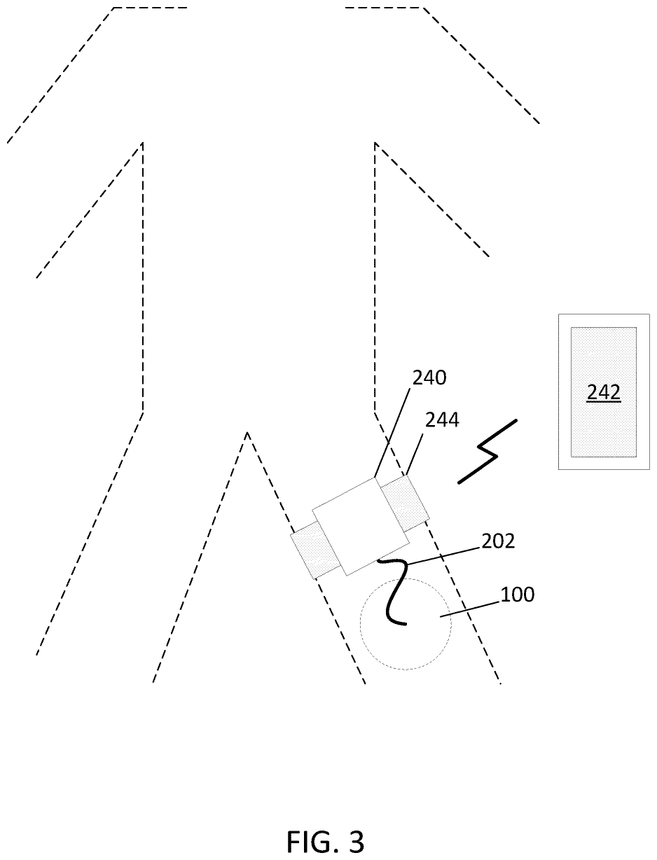

In the example of FIG. 1, the system uses only a single pump 204 that is configured to deliver and/or remove several different fluids to and/or from the closed subcutaneous cavity 100, avoiding the need for multiple pumps that could increase the size, weight, and complexity of the system (which may be a particular issue if used with a wearable system such as the one discussed in the context of FIG. 3 below). In the example of FIG. 1, pump 204 is a reversible pump (e.g. a reversible peristaltic pump or other bi-directional pump) that is in fluid communication with the elongated fluid conduit 202, the irrigation fluid source 206, the medicament source 210, and the fluid outlet container 208.

In the example of FIG. 1, several valves are used in conjunction with pump 204 depending on the operational state of the system. Valve 234 regulates flow of fluid from the irrigation fluid source 206 to the pump 204. Valve 236 regulates flow of medicament from the medicament source 210 to the pump 204. Valve 238 regulates flow of fluid from the pump 204 to the fluid outlet container 208. The system is configured to open and close the valves 234, 236, 238 depending on the operational state of the system. For example, when the system is in a state for pumping irrigation fluid and medicament into the closed subcutaneous cavity 100, the system will operate with open valves 234 and 236, closed valve 238, and operate pump 204 in a direction that will pump fluid from the irrigation fluid source 206 and medicament source 210, through pump 204, through elongated fluid conduit 202, and into the closed subcutaneous cavity 100. If the system is in a state where only irrigation fluid is to be pumped into the closed subcutaneous cavity 100 and not medicament, the system will operate with open valve 234 and closed valves 236 and 238. In operational uses where it may be desirable to vary the volume of medicament that is pumped into the closed subcutaneous cavity 100, mixing valves or other variable state valves may be used in place of valves that are either in a fully open or fully closed state. In some instances, one or more selector valves may be used at intersections of three or more fluid channels.

The system of FIG. 1 also includes an energy source 218 and an antibacterial light source 220. The energy source 218 is configured to generate mechanical, electrical, or ultrasound energy for delivery to the closed subcutaneous cavity 100 to improve fluid flow within the closed subcutaneous cavity 100. In one example implementation, the energy source 218 may be an ultrasound transducer. In some instances, the ultrasound transducer may operate at a frequency in the range of 10 kHz to 1000 kHz, or in the range of 10 kHz to 100 kHz, or in the range of 10 kHz to 50 kHz. Delivery of ultrasound energy to the closed subcutaneous cavity may act to suspend infectious agents in the irrigation fluid so that they are more readily removed from the closed subcutaneous cavity 100. In one example implementation, the antibacterial light source 220 is configured to generate light (e.g. blue light having a frequency or frequencies in the range of between 350 and 450 nm) for delivery to the closed subcutaneous cavity 100 to provide an antibacterial effect. In other implementations the system does not include one or both of the energy source 218 of the antibacterial light source 220.

The system of FIG. 1 also includes a controller 212. The controller is configured to operate the irrigation system in accordance with an irrigation treatment program. In the example of FIG. 1, the controller 212 includes inputs 214 and a display 216 for a user to select an irrigation treatment program. In some implementations, the user may select from several pre-programmed irrigation treatment programs stored in a memory device of the controller 212. In some implementations, the user may input a custom irrigation treatment program into the controller 212. The selected irrigation parameter may include a number of parameters specifying the particular irrigation treatment regimen to be employed.

The irrigation program may include: (i) at least one parameter for pumping an irrigation fluid into the closed subcutaneous cavity 100 from the irrigation fluid source 206, (ii) at least one parameter for dwell time of the irrigation fluid in the closed subcutaneous cavity 100, and (iii) at least one parameter for pumping the irrigation fluid from the closed subcutaneous cavity 100 to the fluid outlet container 208.

Parameters for pumping an irrigation fluid into the closed subcutaneous cavity from the irrigation fluid source may include without limitation: pump direction, duration of pump operation, pump speed, and valve state. Parameters for dwell time of the irrigation fluid in the closed subcutaneous cavity may include without limitation: duration of dwell time, and valve state. Parameters for pumping the irrigation fluid from the closed subcutaneous cavity to the fluid outlet container may include without limitation: pump direction, duration of pump operation, pump speed, and valve state.

The controller 212 may be configured to cause the pump 204 to pump the irrigation fluid from the irrigation fluid source 206 through the elongated fluid conduit 202 and into the closed subcutaneous cavity 100 in accordance with the parameter for pumping the irrigation fluid into the closed subcutaneous cavity 100. The controller 212 may be further configured to allow the irrigation fluid to dwell in the closed subcutaneous cavity 100 in accordance with the parameter for dwell time. The controller 212 may be further configured to cause the pump 204 to pump the irrigation fluid from the closed subcutaneous cavity 100 to the fluid outlet container 208 in accordance with the parameter for pumping the irrigation fluid from the closed subcutaneous cavity.

The irrigation program may include additional parameters regulating operation of the system. Examples of additional parameters include: (i) a parameter or parameters specifying the number of total cycles or a total time for the irrigation treatment program; (ii) a parameter or parameters for pumping the medicament into the closed subcutaneous cavity; (iii) a parameter or parameters governing operation of the energy source 218; and (iv) a parameter or parameters governing operation of the antibacterial light source 220.

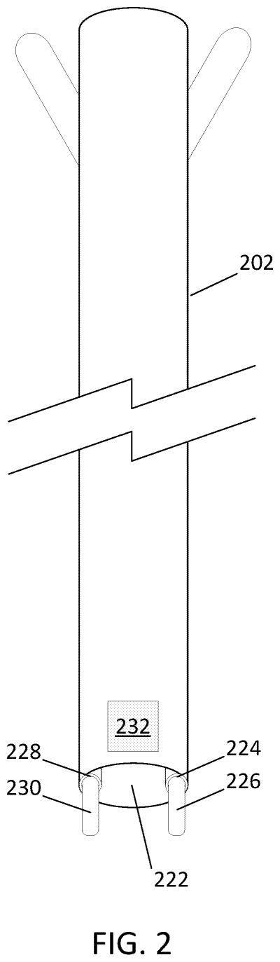

FIG. 2 shows the elongated fluid conduit 202 from FIG. 1 in more detail. In this example, the elongated fluid conduit 202 is a multi-lumen catheter. The elongated fluid conduit 202 includes a main lumen 222. The system is configured to pump the irrigation fluid from the irrigation fluid source 206 and the medicament from the medicament source 210 through the main lumen 222. The system is also configured to pump the irrigation fluid (and other fluids) from the closed subcutaneous cavity 100 to the fluid outlet container 208 through the main lumen 222.

A secondary lumen 224 provides a passageway for an energy transmission member 226 configured to delivery energy from the energy source 218 to the closed subcutaneous cavity 100. Another secondary lumen 228 provides a passageway for a light transmission member 230 (e.g. a fiber optic) configured to deliver antibacterial light from the light source 220 to the closed subcutaneous cavity 100.

In the example of FIG. 2, the elongated fluid conduit 202 includes a pressure sensor 232 for measuring pressure in the closed subcutaneous cavity 100. The system may also include one or more flow meters or other devices for measuring the volume of fluid pumped into and out of the cavity 100.

In some implementations, the system may be configured to quantify the volume of irrigation fluid pumped into and out of the closed subcutaneous cavity 100 and/or measure pressure inside of the closed subcutaneous cavity 100. This information may be used by safety functionality of the system. For instances, exceeding a threshold value of a volume of irrigation fluid in the closed subcutaneous cavity and/or a measured pressure value in the closed subcutaneous cavity may trigger a safety alert, may automatically stop pumping of fluid into the cavity, and/or may open an output valve to allow fluid withdrawal from the cavity. As another example, data about the volume of fluid pumped into and out of the cavity and/or measure pressure values in the cavity may be used in a feedback loop to adjust later cycles of pumping fluid into and out of the cavity. For instance, the system may be configured to adjust the volume of irrigation fluid pumped into and out of the cavity 100 to maintain a target pressure value or target pressure range even as irrigation fluid is cycled through the cavity 100.

FIG. 3 shows an example of an irrigation system in a wearable configuration. In this example, the system includes a wearable unit 240 and a remote controller 242 in wireless communication with the wearable unit 240. In other implementations, controller 242 may be in wired communication with the wearable unit 240. Similar to the example shown in FIG. 1, wearable unit 240 may include a controller, a pump, irrigation fluid source, medicament source, fluid outlet container, an energy source (e.g. ultrasound transducer), and an antibacterial light source. The wearable unit 240 may be configured to facilitate periodic replacement of the irrigation fluid and medicament sources as they become depleted and the fluid outlet container as it becomes full. In some implementations, the irrigation fluid source and the fluid outlet container may have capacities of 1050 mL or less. The wearable unit may also include a rechargeable battery or other power source and a strap 244 or other component for securing the wearable unit on a patient. In some implementations the wearable unit may be configured to be worn around the waist (e.g. a “fanny” pack), as a backpack, as or in a purse or a sling, or as or in a rolling suitcase.

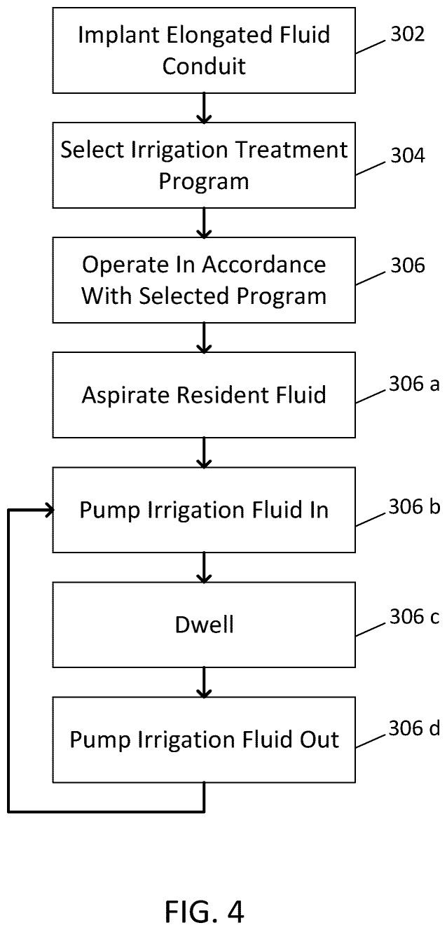

FIG. 4 illustrates an example method of preventing, treating, or otherwise addressing infection risk in a closed subcutaneous cavity with an irrigation system (such as the irrigation systems illustrated in FIGS. 1-3).

At step 302 in FIG. 4, an elongated fluid conduit is implanted into a closed subcutaneous cavity such that the fluid conduit is in fluid communication with the closed subcutaneous cavity. Implantation of the elongated fluid conduit may be performed in a similar fashion to implantation of a typical catheter or surgical drain.

At step 304, an irrigation treatment program is selected. In some implementations, the operator may select from several pre-programmed irrigation treatment programs stored in the irrigation system or may input a custom irrigation treatment program into the system.

Selection of the irrigation treatment program may determine (or at least initially determine) several parameters for the irrigation treatment. These parameters may include, for instance: (i) at least one parameter for pumping an irrigation fluid into the closed subcutaneous cavity, (ii) at least one parameter for dwell time of the irrigation fluid in the closed subcutaneous cavity, (iii) at least one parameter for pumping the irrigation fluid from the closed subcutaneous cavity, (iv) at least one parameter specifying the number of total cycles or a total time for the irrigation treatment program, (v) at least one parameter for delivering a medicament to the closed subcutaneous cavity, (vi) at least one parameter for delivering mechanical, electrical, or ultrasound energy to improve fluid flow within the closed subcutaneous cavity, and (vii) at least one parameter for delivering antibacterial light to the closed subcutaneous cavity.

At step 306, the irrigation system is operated in accordance with the selected irrigation treatment program. In this particular example, operation of the irrigation system includes sub-step 306 a followed by multiple cycles of sub-steps 306b, c, and d.

At sub-step 306 a, resident fluid in the closed subcutaneous cavity is aspirated from the closed subcutaneous cavity. When the method of FIG. 4 is performed using the system of FIG. 1, during sub-step 306 a, the system has closed valves 234 and 236, opened valve 238, and operates pump 204 in a direction that aspirates resident fluid from the closed subcutaneous cavity through the main lumen 222 of elongated fluid conduit 202 and into fluid outlet container 208. During sub-step 306 a, the system may optionally capture or monitor data relating to the volume of resident fluid withdrawn and the pressure in or pressure change in closed subcutaneous cavity 100 and may optionally adjust operating parameters of the selected irrigation treatment program in response to that data.

At sub-step 306 b, the system pumps irrigation fluid into the closed subcutaneous cavity in accordance with the parameter(s) for pumping the irrigation fluid into the closed subcutaneous cavity. When the method of FIG. 4 is performed using the system of FIG. 1, during sub-step 306 b, the system has opened valve 234 and closed valve 238 and operates pump 204 in a direction (opposite to the pumping direction of sub-step 306 a) that pumps irrigation fluid from the irrigation fluid source 206 through the main lumen 222 of elongated fluid conduit 202 and into the closed subcutaneous cavity 100. Depending on the parameters of the selected irrigation treatment program, the system may also have opened valve 234 such that medicament is pumped from medicament source 210, through the main lumen 222 of the elongated fluid conduit 202, and into the closed subcutaneous cavity 100 simultaneously with the irrigation fluid. In other implementations, the system may pump medicament into the closed subcutaneous cavity in a separate step from irrigation fluid pumping. During sub-step 306 b, the system may optionally capture or monitor data relating to the volume of irrigation fluid pumped into the closed subcutaneous cavity 100 and the pressure in or pressure change in closed subcutaneous cavity 100 and may optionally adjust operating parameters of the selected irrigation treatment program in response to that data. For instance, in one implementation, the system may be configured to reduce the volume of irrigation fluid pumped into the closed subcutaneous cavity when the pressure in the cavity is above a certain threshold.

In some implementations, during sub-step 306 b, the irrigation fluid is pumped into the closed subcutaneous cavity at a flow rate that is less than 20 mL per second, or less than 10 ml per second and at outlet pressures that are less than 200 pounds per square inch or less than 100 pounds per square inch.

At sub-step 306 c, the irrigation fluid is allowed to dwell in the closed subcutaneous cavity in accordance with the parameter(s) for dwell time. During sub-step 306 c, the system may optionally capture or monitor data relating to pressure in or pressure change in closed subcutaneous cavity 100 and may optionally adjust operating parameters of the selected irrigation treatment program in response to that data.

At sub-step 306 d, the system pumps irrigation fluid from the closed subcutaneous cavity in accordance with the parameter(s) for pumping the irrigation fluid into the closed subcutaneous cavity. When the method of FIG. 4 is performed using the system of FIG. 1, during sub-step 306 d, the system has closed valves 234 and 236 and opened valve 238 and operates pump 204 in a direction (opposite to the pumping direction of sub-step 306 b) that pumps irrigation fluid out of the closed subcutaneous cavity 100 through the main lumen 222 of elongated fluid conduit 202 and into the fluid outlet container 208. During sub-step 306 d, the system may optionally capture or monitor data relating to the volume of irrigation fluid withdrawn and the pressure in or pressure change in closed subcutaneous cavity 100 and may optionally adjust operating parameters of the selected irrigation treatment program in response to that data.

After sub-step 306 d, the system may execute additional cycles of sub-steps 306 b, c, and d in accordance with selected parameters for total cycles or total time for the irrigation treatment program. In some uses, total time may exceed eight or even exceed twenty four hours.

During any of the aforementioned steps, or at separate steps between one of the aforementioned steps, the system may deliver antibacterial light and/or mechanical, electrical, or ultrasound energy to the closed subcutaneous cavity.

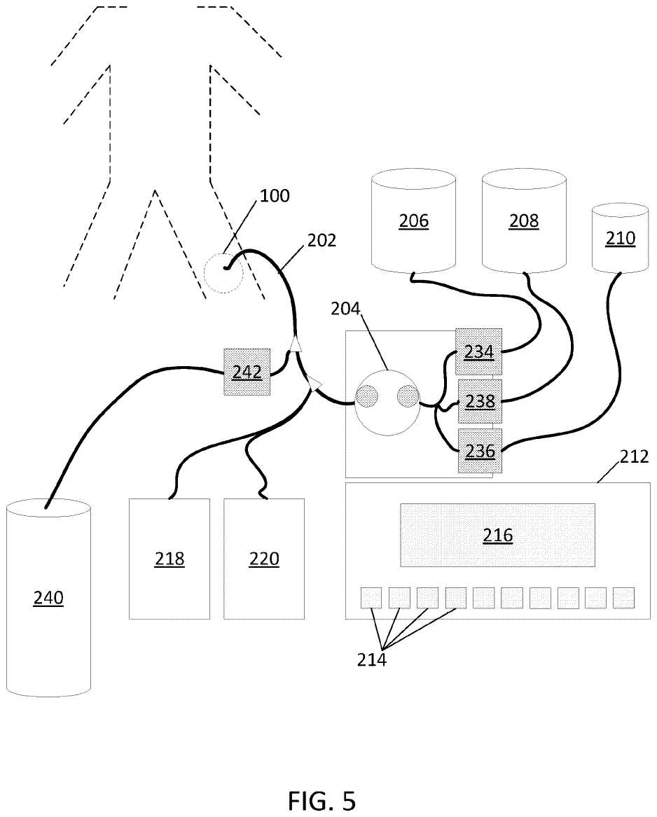

FIG. 5 illustrates another example of irrigation system for preventing, treating, or otherwise addressing infection risk in a closed subcutaneous cavity 100. In the example of FIG. 1 all flow of irrigation fluid into and out of the closed subcutaneous cavity 100 goes through pump 204. In that example, when the pump 204 is not pumping, irrigation fluid and other fluids in the cavity 100 cannot flow out of the closed subcutaneous cavity 100 through the elongated fluid conduit 202. FIG. 5 illustrates an alternative configuration that allows for passive drainage as part of a prescribed treatment plan.

In the example of FIG. 5 a passive drainage outlet 240 is in fluid communication with the elongated fluid conduit 202 between the distal end of the elongated fluid conduit 202 (where it is implanted in closed subcutaneous cavity 100) and the pump 204. Passive drainage valve 242 is in fluid communication with the passive drainage outlet 240, with the controller 212 configured to cause the passive drainage valve 242 to be in an open state when the system is in a passive drainage condition. The controller 212 may further be configured to cause the passive drainage valve 242 to be in a closed state while the pump 204 pumps irrigation fluid into and from the closed subcutaneous cavity 100. Parameters may be included in the controller 212 programming for the passive drainage condition (e.g. preset times or other criteria for entering the passive drainage condition, duration of the passive drainage condition, etc.).

When the system is in the passive drainage condition fluid may drain from closed subcutaneous cavity 100 into the passive drainage outlet 240. Passive drainage outlet 240 may be truly “passive” in the sense that only gravity is required for fluid to drain from closed subcutaneous cavity 100 into the passive drainage outlet 240. In other implementations, passive drainage outlet 240 may be a bulb cannister or other device capable of creating a negative pressure environment to encourage drainage from the closed subcutaneous cavity 100 without operation of the pump 204.

### Example Use Cases

The systems and methods described above can be placed and implemented in various clinical scenarios, including as a wearable, at-home product, in the clinic, in an interventional radiology suite, or in the operating room to treat a variety of types of joint infection. Examples of target patient populations include patients with swelling surrounding their joint replacements, infected native joints, and infected seromas. Additionally, the systems and methods described above can be used to treat critically ill patients who are unable to undergo a formal surgical washout. Additionally, the systems and methods described above may also be used as a preventative post-operative treatment. We anticipate the above described systems and methods should improve efficacy of initial washout and antibiotic treatments, decrease costly hospital stays, and open new opportunities for non-OR use.

### Prospective Example 1

A patient presents to the Emergency Department with a knee effusion. Previous knee replacement performed at another hospital out-of-state. Ortho is consulted and the knee is aspirated. Fluid is sent off and the patient admitted for IV antibiotics. Microbiology analysis demonstrates infection. Patient taken to the OR in 1-2 days for irrigation, debridement, and exchange of parts. Surgical drain is placed and patient is kept on IV antibiotics for 6 weeks. Persistent cloudy drainage noted. No additional washouts performed. In this prospective example, the systems and methods described above could be utilized postop to provide additional washouts and deliver antibiotics directly to the site of infection to improve efficacy of initial treatments.

### Prospective Example 2

A patient presents to the Emergency Department with a knee effusion. Surgical history significant for knee replacement complicated by infection requiring surgical washout. Ortho is consulted and the knee is aspirated again. Fluid is sent off and patient admitted for IV antibiotics. Microbiology analysis demonstrates repeat infection. Patient taken to the OR 3 days later for irrigation, debridement, removal of hardware and placement of antibiotic impregnated cement spacer. Surgical drain is placed for 3 days and patient is kept on IV antibiotics for 6 weeks. Patient brought back to OR 2 months later for removal of spacer and revision joint replacement. Intraop evaluation with gram stain reveals persistent infection. A second cement spacer is placed and additional 2 months of IV antibiotics. Patient taken back to the OR for a third time and cement spacer removed and revision joint replacement performed. In this prospective example, the systems and methods described above could have been placed postop to provide additional washouts and deliver antibiotics directly to the site of infection to improve efficacy of initial treatments.

### Prospective Example 3

A patient presents to the clinic with a knee effusion. Knee is aspirated and cloudy fluid noted. Patient given prescription for antibiotics and sent home with instructions to return if symptoms worsen. Effusion worsens and patient returns to ED next day with increased pain. Patient admitted and started on IV antibiotics. Knee is re-aspirated and fluid sent for cultures again. 1st set of cultures positive for infection. Patient taken to OR for arthroscopic washout and drain placement. In this prospective example, a wearable embodiments of the above described systems and methods could have been placed during the initial clinic encounter after aspiration was performed. This would have provided continuous cycles of irrigation and aspiration while waiting on cultures. Once culture results became available an antibiotic infusion could have been tailored based on the culture specificity.

### Prospective Example 4

A female with breast cancer was involved in a motor vehicle accident and sustained a large open wound on the outer right thigh and knee. Treated at an outside hospital with surgical debridement and wound closure. Follow-up with outside hospital surgeon 1 week prior to presentation at our hospital—patient voiced concerns for infection due to swelling along the thigh and drainage. Surgeon was not concerned. Patient presented to our facility after increased swelling and drainage. Imaging demonstrated a fluid collection with a differential of seroma versus abscess. Fluid aspirated and microbiology results were positive for MRSA. Required 2nd surgical debridement and placement of surgical drain. Continued murky drainage noted for days after surgery. Consequently, there were multiple admissions and lengthy hospital stays. Patient may require additional surgical washouts. In this prospective example, the above described systems and methods could have been applied for continuous irrigation and antibiotic delivery as soon as swelling was noted in the area. This would have likely resolved the infection without the need for additional surgical procedures. A wearable version of the above described systems and methods could also decrease costly inpatient hospital stays.

### Prospective Example 5

Critically ill patients may have abscesses or joint infections causing sepsis, however these patients may be too unstable to undergo a formal surgical washout. Serial aspirations are often performed at the bedside with or without IR drain placement. These temporizing measures have limited efficacy. In this prospective example, the above described systems and methods could be an ideal solution for this patient population to provide continuous irrigation and antibiotic delivery at the bedside.

### Prospective Example 6

Prophylactic use of vancomycin powder placed in spine wounds has been shown to decrease the incidence of infection. However, there is an increase in the incidence of postoperative seromas with the use of vancomycin powder. The proposed mechanism is vancomycin powder becoming crystalized as a salt with hydrochloride, which creates an osmotic gradient when placed in the surgical wound. Persistent seromas may lead to patient discomfort and increased risk for late infection. In this prospective example, the above described systems and methods could be used for high-risk surgical wounds to prevent postop infections and prevent seroma formation.

### Additional Prospective Use Notes

The above described systems could be used with standard surgical drains or specialized catheters; could be placed at the bedside, in the clinic, in an interventional radiology suite or in the OR; could be used on multiple anatomic sites (knee, shoulder, hip, thigh, calf, pelvis, paraspinal); may decrease length of hospital stay and need for multiple surgical procedures; may include multimodal therapy such as blue light and sonification; may include machine learning algorithms that personalize treatment regimens to patient volume capacities and pressure sensitivities; may be programmable and antibiotic therapy may be tailored according to future culture results.

### Evaluation for Cross-Contamination Risk

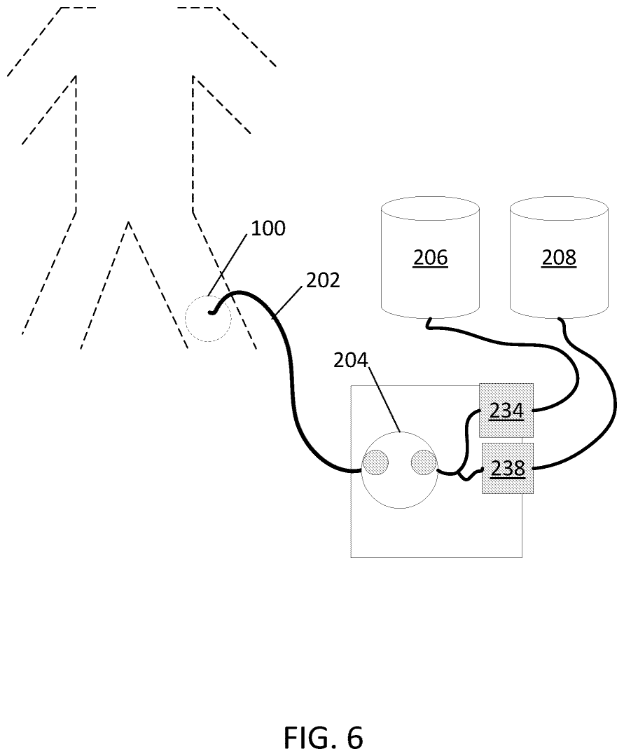

The inventors have evaluated the efficacy of the above-described systems and methods in preventing cross-contamination during push/pull cycles of the system. FIG. 6 schematically shows the configuration of the system for this evaluation. For this evaluation a push/pull cycle involves drawing approximately 50 ml of input liquid from fluid source 206, delivering it into a closed subcutaneous cavity 100 for approximately 16 seconds, and subsequently retrieving the liquid from the closed subcutaneous cavity 100 and depositing it into fluid outlet container 208. A single pump 204 facilitated both the input and output functions, with fluid source valve 234 in an open state and fluid outlet valve 238 in a closed state during the push, and valve fluid source valve 234 in a closed state and fluid outlet valve 238 in an open state during the pull.

The inventors wished to investigate how proficient the valving system would be in maintaining separation between the fluid source 206 and fluid outlet 208 during push/pull cycles that used a common pump 204 and common fluid conduit 202. The inventors performed the evaluation by measuring pH level using a litmus test to ascertain whether the clean fluid source 206 would become contaminated after undergoing multiple push/pull cycles.

A protocol was devised involving measuring pH at the fluid source 206, fluid outlet 208, and closed subcutaneous cavity 100 using litmus paper, and testing it through multiple push/pull cycles. A standard pH litmus scale was used to determine the liquid's pH based on the color of the litmus test strip after being dipped in the liquid.

Two tests were conducted, one to establish a baseline with no intentional contamination and another with a deliberate change in pH within the joint. These tests aided in understanding the liquid behavior within the system and its capability to clean the closed subcutaneous cavity 100 without cross-contamination.

Each test involved measuring the pH of extracted liquid from the fluid source 206, fluid outlet 208, and closed subcutaneous cavity 100. Sterile water with a pH of 7.0 was used as the fluid source liquid in this experiment.

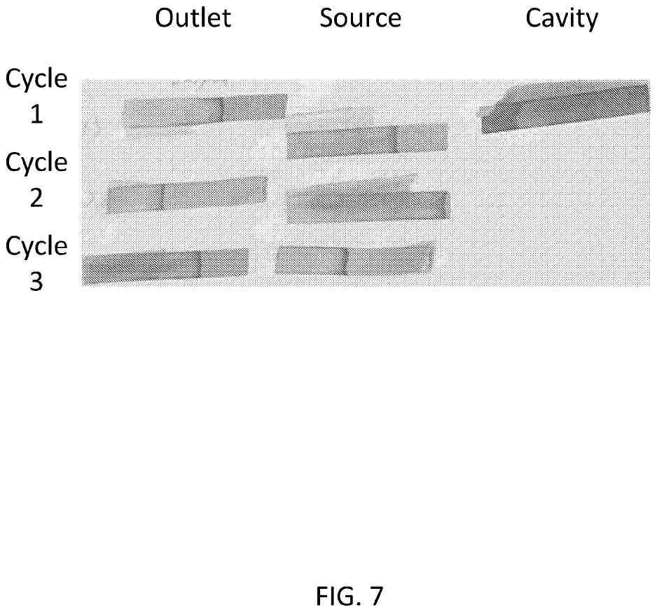

To establish a baseline measurement of extracted liquid from the fluid source 206, fluid outlet 208, and closed subcutaneous cavity 100, a litmus test was performed. Then, a push/pull cycle was run, and the pH was measured at each location. This process was repeated twice. The pH in the output container was then visually compared with the original pH observed in the knee. The pH results of the litmus test strips are shown in FIG. 7.

For the intentional contamination test, betadine was used as a pH change agent due to its pH characteristics and distinctive color, facilitating the observation of the clearing process within the cavity 100. Considering the properties of the tissue, an agent capable of altering the pH by 3 units was necessary to modify the joint environment effectively. This choice enabled assessment of cross-contamination of the system while minimizing tissue denaturation or damage, thereby ensuring accurate reflection of joint behavior in a clinical setting.

To start the intentional pH change test, 10 mL of the pH change agent (betadine) was injected into the cavity. Then the pH of the fluid source 206, fluid outlet 208, and closed subcutaneous cavity 100 was measured after a push/pull cycle. The pH in the fluid outlet 208 was then compared with the original pH observed in the cavity 100. This process was repeated six times for the intentional pH contamination test.

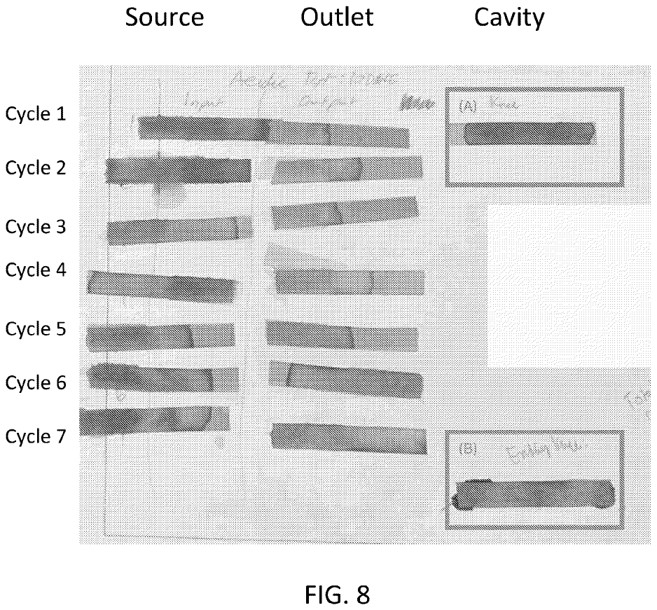

In the intentional contamination test, after seven cycles, the pH of the cavity 100 was reassessed to determine if the system effectively cleaned the joint cavity 100. The pH results of the litmus test strips for this test are shown in FIG. 8.

In the intentional pH change test results (FIG. 8), the initial pH of the knee joint cavity was measured at 4.0 (Label A). However, by the conclusion of the 7th cycle, the pH had risen to approximately 7.0 (Label B), aligning with both the source and outlet pH levels and matching the baseline pH observed before testing commenced.

Throughout the cycles, the pH of the source liquid remained consistent, showing no decrease, indicating minimal to no betadine infiltration into the sterile water supply. This observation was corroborated by the absence of color change in the IV bag and tubing used for the source for the testing. Similarly, the pH of the outlet remained stable around 7 throughout the 7 cycles. However, there was a significant change in the color of the liquid in the outlet container, attributed to the presence of betadine from the joint, indicating successful cleansing of the joint by the system with minimal risk of cross-contamination.

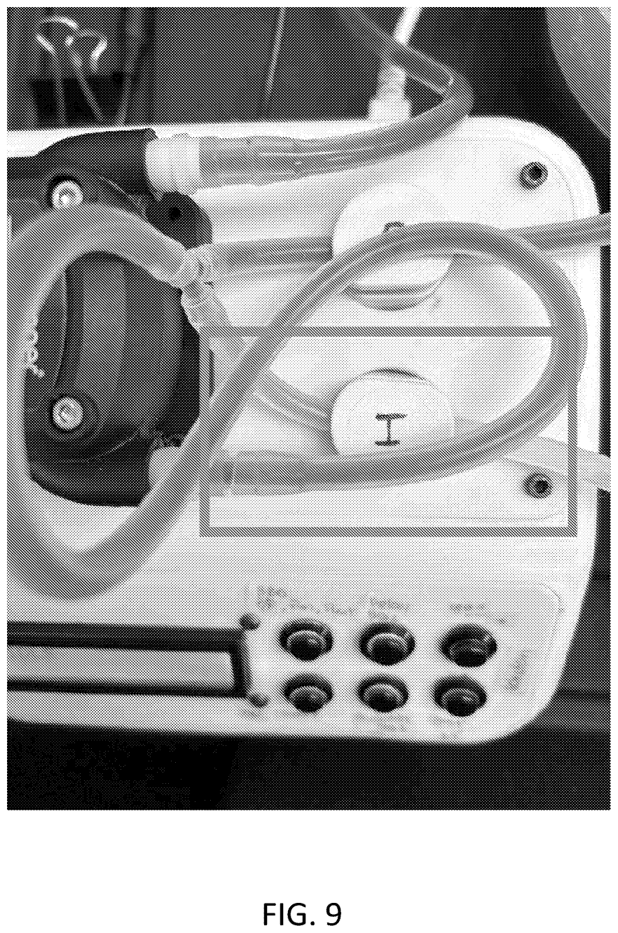

During testing, it was noted that there was minimal backflow observed in the pull phase of the cycle. However, liquids from the joint cavity consistently flowed toward the source tube without crossing into the source itself throughout all 7 cycles, indicating that the valve system effectively prevented such cross-contamination within the system, as shown in FIG. 9.

Following the pH testing, additional push/pull cycles were conducted to determine the optimal number required for satisfactory joint cavity clearance. By the 8th cycle, the fluid removed from the cavity appeared nearly transparent with minimal traces of betadine. However, it was acknowledged that this outcome might vary in the future, as it was suggested to potentially reduce the liquid volume transported through the joint per cycle, thereby necessitating more cycles for complete joint cavity clearance.

In conclusion, the results demonstrated by the pH measurements and visual observation of the betadine in the tubing indicate a notable absence of liquid crossover or backflow into the input fluid source, thereby demonstrating little to no cross-contamination occurs while operating the system.

### End Notes

Additions, deletions, substitutions, and other modifications may be made to the example systems and methods described above without departing from the scope or the spirit of the following claimed inventions.

1. An irrigation system for addressing infection risk in a closed subcutaneous cavity, the irrigation system comprising:
(a) an elongated fluid conduit configured for implantation in a closed subcutaneous cavity;
(b) a pump configured to pump an irrigation fluid through the elongated fluid conduit into and from the closed subcutaneous cavity;
(c) an irrigation fluid source;
(d) a fluid source valve located along a fluid path between the irrigation fluid source and the pump; and
(e) a controller configured to operate the irrigation system, wherein the controller is configured to cause the fluid source valve to be in an open state while the pump pumps the irrigation fluid from the irrigation fluid source through the fluid conduit, and wherein the controller is configured to cause the fluid source valve to be in a closed state while the pump pumps the irrigation fluid from the closed subcutaneous cavity.

2. The irrigation system of claim 1 wherein:
(a) the controller is configured to operate the irrigation system in accordance with an irrigation treatment program, the irrigation treatment program comprising: (i) at least one parameter for pumping an irrigation fluid into the closed subcutaneous cavity, (ii) at least one parameter for dwell time of the irrigation fluid in the closed subcutaneous cavity, and (iii) at least one parameter for pumping the irrigation fluid from the closed subcutaneous cavity;
(b) the controller is configured to cause the pump to pump the irrigation fluid from the irrigation fluid source through the fluid conduit and into the closed subcutaneous cavity in accordance with the parameter for pumping the irrigation fluid into the closed subcutaneous cavity;
(c) the controller is configured to allow the irrigation fluid to dwell in the closed subcutaneous cavity in accordance with the parameter for dwell time; and
(d) the controller is configured to cause the pump to pump the irrigation fluid from the closed subcutaneous cavity in accordance with the parameter for pumping the irrigation fluid from the closed subcutaneous cavity.

3. The irrigation system of claim 2 further comprising an irrigation fluid outlet, wherein the controller is configured to cause the pump to pump the irrigation fluid from the closed subcutaneous cavity through the fluid conduit and into the irrigation fluid outlet in accordance with the parameter for pumping the irrigation fluid from the closed subcutaneous cavity.

4. The irrigation system of claim 3 further comprising a fluid outlet valve located along a fluid path between the irrigation fluid outlet and the pump; wherein the controller is configured to cause the fluid outlet valve to be in an open state while the pump pumps the irrigation fluid from the closed subcutaneous cavity.

5. The irrigation system of claim 4 wherein the controller is configured to cause the fluid outlet valve to be in a closed state while the pump pumps the irrigation fluid from the irrigation fluid source through the fluid conduit.

6. The irrigation system of claim 5 wherein the irrigation fluid source is an irrigation fluid source container and where the irrigation fluid outlet is an irrigation fluid outlet container.

7. The irrigation system of claim 3 wherein the elongated fluid conduit comprises a distal end configured for implantation in the closed subcutaneous cavity; and wherein the irrigation system further comprises a passive drainage outlet in fluid communication with the elongated fluid conduit between the distal end of the elongated fluid conduit and the pump.

8. The irrigation system of claim 7 further comprising a passive drainage valve in fluid communication with the passive drainage outlet, wherein the controller is configured to cause the passive drainage valve to be in an open state when the irrigation system is in a passive drainage condition.

9. The irrigation system of claim 8 wherein the controller is configured to cause the passive drainage valve to be in a closed state while the pump pumps the irrigation fluid from the closed subcutaneous cavity; and
wherein the controller is configured to cause the passive drainage valve to be in a closed state while the pump pumps the irrigation fluid from the irrigation fluid source through the fluid conduit.

10. The irrigation system of claim 8 wherein the irrigation treatment program comprises at least one parameter for passive drainage of the closed subcutaneous cavity.

11. The system of claim 2, wherein the controller includes at least one input for selecting the irrigation treatment program.

12. The system of claim 11, wherein the input is configured to allow selection of the irrigation treatment program from a plurality of pre-programmed irrigation treatment programs.

13. The system of claim 11, wherein the input is configured to allow input of a custom irrigation treatment program.

14. The system of claim 11, wherein the irrigation treatment program further comprises at least one parameter specifying the number of total cycles or a total time for the irrigation treatment program.

15. The system of claim 11, further comprising a medicament source, wherein the controller is configured to cause the pump to pump a medicament from the medicament source through the fluid conduit and into the closed subcutaneous cavity in accordance with a parameter for pumping the medicament into the closed subcutaneous cavity.

16. The system of claim 11, wherein the fluid conduit is a multi-lumen catheter including a first lumen and a second lumen.

17. The system of claim 16, wherein the system is configured to pump the irrigation fluid and the medicament into the closed subcutaneous cavity through the first lumen and to pump the irrigation fluid out of the closed subcutaneous cavity through the first lumen.

18. The system of claim 16, wherein a energy transmission member extends through the second lumen.

19. The system of claim 18, further comprising an ultrasound transducer connected to the energy transmission member.

20. The system of claim 16, wherein a light transmission member extends through the second lumen.

21. The system of claim 20, further comprising a light source connected to the light transmission member, the light source configured to supply light having a wavelength or wavelengths in the range of 350 nm to 450 nm to the light transmission member.

22. The system of claim 11, wherein pump, the irrigation fluid source, and the controller are contained in a wearable pumping unit, the wearable pumping unit further comprising a drain container configured to receive irrigation fluid pumped from the closed subcutaneous housing, the wearable pumping unit further comprising an energy source configured to power the pump.

23.-67. (canceled)

68. An irrigation system for addressing infection risk in a closed subcutaneous cavity comprising:
(a) an elongated fluid conduit configured for implantation in a closed subcutaneous cavity;
(b) a pump configured to pump an irrigation fluid through the elongated fluid conduit;
(c) an irrigation fluid source; and
(d) a controller configured to operate the irrigation system in accordance with an irrigation treatment program, the irrigation treatment program comprising: (i) at least one parameter for pumping an irrigation fluid into the closed subcutaneous cavity, (ii) at least one parameter for dwell time of the irrigation fluid in the closed subcutaneous cavity, and (iii) at least one parameter for pumping the irrigation fluid from the closed subcutaneous cavity;
(e) wherein the controller is configured to cause the pump to pump the irrigation fluid from the irrigation fluid source through the fluid conduit and into the closed subcutaneous cavity in accordance with the parameter for pumping the irrigation fluid into the closed subcutaneous cavity;
(f) wherein the controller is configured to allow the irrigation fluid to dwell in the closed subcutaneous cavity in accordance with the parameter for dwell time; and
(g) wherein the controller is configured to cause the pump to pump the irrigation fluid from the closed subcutaneous cavity in accordance with the parameter for pumping the irrigation fluid from the closed subcutaneous cavity.
